(19) United States
(12) Patent Application Publication
(10) Pub. No.: US 2004/0199030 A1
(43) Pub. Date: Oct. 7, 2004

(54) Process for production of high-temperature and high-pressure fluid and high-temperature and high-pressure reaction system

(76) Inventors: Kiyotaka Hatakeda, Miyagi (JP); Osamu Sato, Miyagi (JP); Yutaka Ikushima, Miyagi (JP); Kazuo Torii, Miyagi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,812

(22) PCT Filed: Sep. 27, 2002
(86) PCT No.: PCT/JP02/10096

(30) Foreign Application Priority Data
Sep. 27, 2001 (JP) 2001-295965

(51) Int. Cl. C07C 27/00

(52) U.S. Cl. 568/959

(57) ABSTRACT

The present invention provides a method for the production of a high-temperature high-pressure fluid which can be caused to reach specified conditions in a short time, a high-temperature high-pressure reaction method, and a reaction system for the same, and the present invention comprises a method for the production of a high-temperature high-pressure fluid in which the reactants can be caused to reach a prescribed temperature in 5 seconds or less by mixing two or more high-pressure fluids at different temperatures in a flow system, a high-temperature high-pressure reaction method which utilizes this production method of a high-temperature high-pressure fluid, and which reduces the temperature elevation time to the prescribed reaction temperature to 5 seconds or less by mixing a carrier fluid at a temperature higher than the prescribed reaction temperature with substrate solution(s) at a temperature of 100° C. or lower and reacting these fluids inside a reaction vessel, thus suppressing side reactions that occur during the temperature elevation and making it possible to perform short-time chemical reactions efficiently and selectively, and a reaction system for this reaction method.

### BACKGROUND OF THE INVENTION

1. Field of the Invention

The present invention relates to a method for the production of a high-temperature high-pressure fluid and a high-temperature high-pressure reaction method and reaction system utilizing this method. More particularly, the present invention relates to a method for producing a high-pressure fluid by mixing high-pressure fluids at different temperatures in a flow system, a high-temperature high-pressure reaction method in which target compounds are produced by reacting reactants at a high temperature and high pressure in a flow system utilizing this production method, and in which various types of production, reactions and the like can be performed in a short time in a high-temperature high-pressure fluid such as a supercritical fluid or the like, and a reaction system for this reaction method.

2. Description of the Related Art

Methods for producing high-temperature high-pressure fluids include two types of methods, i.e., batch systems in which a high-temperature high-pressure fluid is produced using a sealed vessel such as an autoclave or the like, and flow systems in which high-temperature high-pressure water is produced through heating by means of a heater or the like. Both types of methods suffer from the drawback of requiring considerable time in order to reach the prescribed temperature. Furthermore, methods used to produce substances at a high temperature and high pressure can be broadly classified into two types of methods, i.e., batch systems and flow systems. For example, reaction apparatuses for supercritical water belong either to the category of conventional batch type apparatuses which use a reaction apparatus such as an autoclave or the like, or to the category of flow type apparatuses. In the past, in both types of reaction apparatuses, induction to the prescribed target temperature has been accomplished by a method in which water at room temperature or an aqueous solution formed by added reactant raw materials to such water is elevated to the desired prescribed temperature by means of an electric furnace or molten salt bath set at a high temperature.

These methods suffer from the following drawback: namely, a time of approximately 20 seconds to 10 hours is ordinarily required in order to reach the prescribed reaction temperature from room temperature. However, such methods have been used in reactions which are such that side reactions do not occur during temperature elevation, or which are such that side reactions occurring during temperature elevation can be ignored, as indicated for example in methods in which harmful organic substances are subjected to oxidative decomposition in supercritical water at 400° C. and 30 MPa or greater (Dong-Soo Lee and Earnest F. Gloyana, Jour. Supercritical Fluids, Vol. 3, 249-255 (1990)), methods for the water-heated synthesis of minerals or the like.

In many cases, however, such conventional techniques cannot be used in organic synthesis reactions which are such that side reactions occur during the elevation of the temperature from room temperature to the prescribed reaction temperature. For example, in cases where a reaction in which ε-caprolactam is produced by a conventional method from cyclohexanoneoxime through a Beckman transfer reaction using high-temperature high-pressure water, the temperature must be elevated to a temperature of 200 to 400° C. in 20 to 30 seconds in the case of a batch method using a molten salt bath. As a result, a hydrolysis reaction from cyclohexanoneoxime to cyclohexanone occurs, so that the yield of the desired ε-caprolactam is a low value (O. Sato, Y. Ikushima and T. Yokyoyama, Jour. Organic Chemistry, Vol. 63, 9100-9102 (1998)).

Meanwhile, a method for synthesizing ε-caprolactam from cyclohexanoneoxime by a flow system that has been used in the past (Y. Ikushima, K. Hatakeda, O. Sato, T. Yokoyama and M. Arai, Jour. Am. Chem. Soc., Vol. 122, 1908-1918 (2000)) uses high-temperature high-pressure water whose temperature is elevated from room temperature. In an experiment in which a reaction was performed for 113 seconds at a temperature of 350° C. and a pressure of 22.1 MPa, only cyclohexanone produced by a hydrolysis reaction was obtained. On the other hand, in an experiment performed at a temperature of 374.5° C., ε-caprolactam and cyclohexanone were produced, and a hydrolysis reaction of cyclohexanoneoxime occurred during the elevation of the temperature, so that this method appeared to suffer from the drawback of a drop in the yield of the target ε-caprolactam. The flow system is a method suited to mass production; however, if the temperature elevation time is not shortened, this method cannot be applied to organic synthesis reactions, so that there is a demand for technical improvement.

### SUMMARY OF THE INVENTION

Under such circumstances, the present inventors, in light of the abovementioned prior art, have discovered that it is important to reach the prescribed reaction temperature in a short time in order to prevent the occurrence of side reactions in synthesis reactions of organic compounds such as the production of ε-caprolactam from cyclohexanoneoxime under high-temperature high-pressure conditions or the like. The present inventors perfected the present invention by conducting further research on the basis of this finding. Specifically, it is one object of the present invention to provide a method for producing a high-temperature high-pressure fluid in a short time of 5 seconds or less. Furthermore, it is another object of the present invention to provide a high-temperature high-pressure reaction method which makes it possible to cause the reactants to reach the prescribed reaction temperature in 3 seconds or less, and a reaction system using this method.

The present invention, which is used to solve the abovementioned problems, is constructed form the following technical means.

(1) A method for the production of a high-temperature high-pressure fluid by mixing two or more high-pressure fluids at different temperatures in a flow system, wherein the reactants are caused to reach the prescribed reaction temperature in 5 seconds or less by mixing a high-pressure fluid having a temperature higher than the prescribed temperature with a high-pressure fluid having a temperature lower than the prescribed temperature.

(2) The method for the production of a high-temperature high-pressure fluid according to (1), wherein a high-temperature high-pressure fluid in a temperature range of 250 to 600° C. and a pressure range of 10 to 100 MPa is produced.

(3) The method for the production of a high-temperature high-pressure fluid according to (1) or (2), wherein one or more high-temperature high-pressure fluids selected from a group comprising water, acetonitrile, ethyl alcohol, acetone, dimethyl sulfoxide, 1,4-dioxane, N,N-dimethylformamide and tetrahydrofuran are used and/or produced.

(4) A high-temperature high-pressure reaction method for producing target substances by reacting one or more reactants in a high-temperature high-pressure fluid in a flow system, wherein the reactants are caused to reach the prescribed reaction temperature in 5 seconds or less by feeding a high-pressure fluid at a higher temperature than the prescribed reaction temperature into a reaction vessel at a high speed as a carrier fluid, injecting one or more substrate high-pressure fluids which contain reactants and which have a lower temperature than the prescribed reaction temperature into the reaction vessel at a speed lower than the abovementioned speed, and mixing these fluids.

(5) The high-temperature high-pressure reaction method according to (4), wherein the substances to be treated are reacted in a temperature range of 250 to 600° C. and a pressure range of 10 to 100 MPa.

(6) The high-temperature high-pressure reaction method according to (4) or (5), wherein a high-pressure fluid at a temperature of 5 to 400° C. higher than the prescribed reaction temperature is used as the carrier fluid.

(7) The high-temperature high-pressure reaction method according to any one of the abovementioned (4) through (6), wherein the temperature of the substrate high-pressure fluid containing the reactants is 100° C. or lower.

(8) The high-temperature high-pressure reaction method according to any one of the abovementioned (4) through (7), wherein the linear velocity of the carrier fluid and/or substrate high-pressure fluid is 10⁻⁶ to 10³ m/sec.

(9) The high-temperature high-pressure reaction method according to any one of (4) through (8), wherein the value of the linear velocity of the substrate high-pressure fluid containing the reactants is in the range of 0.0001 to 1, where the linear velocity of the carrier fluid is 1.

(10) The high-temperature high-pressure reaction method according to any one of (4) through (9), wherein the feeding rate of the carrier fluid and/or substrate high-pressure fluid is 10⁻³ to 10⁶ ml/min.

(11) The high-temperature high-pressure reaction method according to any one of (4) through (10), wherein the value of the feeding rate of the substrate high-pressure fluid containing reactants is a value in the range of 0.0001 to 1, where the feeding rate of the carrier fluid is 1.

(12) The high-temperature high-pressure reaction method according to any one of (4) through (11), wherein the reaction time is 30 seconds or less.

(13) The high-temperature high-pressure reaction method according to any one of (4) through (12), wherein one or more high-temperature high-pressure fluids selected from a group comprising water, acetonitrile, ethyl alcohol, acetone, dimethyl sulfoxide, 1,4-dioxane, N,N-dimethylformamide and tetrahydrofuran is used as a fluid.

(14) A high-temperature high-pressure reaction system for use in the reaction method according to any one of (4) through (13), and for producing target substances by reacting one or more reactants in a high-temperature high-pressure fluid in a flow system, comprising: means for feeding a high-pressure fluid having a higher temperature than the prescribed reaction temperature into a reaction vessel as a carrier fluid; and means for injecting one or more low-temperature substrate high-pressure fluids containing reactants into the reaction vessel, wherein the reactants are caused to reach the prescribed reaction temperature in 5 seconds or less by feeding a high-pressure fluid at a higher temperature than the prescribed reaction temperature into a reaction vessel at a high speed as a carrier fluid, injecting one or more substrate high-pressure fluids which contain reactants and which have a lower temperature than the prescribed reaction temperature into the reaction vessel at a speed lower than the abovementioned speed, and mixing these fluids.

The present inventors have perfected the present invention as a result of conducting diligent research in an attempt to shorten the temperature elevation time in a flow type apparatus, in which a time of 20 seconds or longer is required in order to elevate the temperature to the prescribed temperature in the case of a conventional high-temperature high-pressure reaction apparatus. Specifically, one aspect of the present invention is a method for the production of a high-temperature high-pressure fluid by mixing two or more high-pressure fluids at different temperatures in a flow system, wherein the reactants are caused to reach a prescribed temperature in 5 seconds or less by mixing a high-pressure fluid which is at a higher temperature than the prescribed temperature with a high-pressure fluid which is at a lower temperature than the prescribed temperature. Furthermore, other aspects of the present invention include a high-temperature high-pressure reaction method for producing target substances by reacting one or more reactants in a high-temperature high-pressure fluid in a flow system, wherein the reactants are caused to reach the prescribed reaction temperature in 5 seconds or less by feeding a high-pressure fluid which is at a higher temperature than the prescribed reaction temperature into a reaction vessel at a high speed as a carrier fluid, injecting one or more substrate high-pressure fluids which contain reactants and which are at a lower temperature than the abovementioned prescribed reaction temperature into the reaction vessel at a speed that is lower than the abovementioned speed, and mixing these fluids, and a high-temperature high-pressure reaction system which is used in this reaction method, wherein this reaction system comprises means for feeding a high-pressure fluid which is at a higher temperature than the prescribed reaction temperature into the reaction vessel as a carrier fluid, and means for injecting one or more low-temperature substrate high-pressure fluids containing reactants into the reaction vessel.

The method of the present invention for the production of a high-temperature high-pressure fluid will be described below; then, the high-temperature high-pressure reaction method of the present invention and the corresponding reaction system will be described.

In the present invention, a high-temperature high-pressure fluid at a prescribed temperature can be produced in a short time of 5 seconds or less by using a high-pressure fluid which is at a higher temperature than the prescribed reaction temperature as a carrier fluid, and mixing a high-pressure fluid which is at a lower temperature than the prescribed temperature with this carrier fluid. The prescribed temperature of a high-temperature high-pressure fluid may conceivably be controlled by varying the size, volume and shape of the reaction vessel, the type of the high-pressure fluid which is at a higher temperature than the prescribed temperature, and the type, temperature, pressure, feeding rate, linear velocity and the like of the high-pressure fluid which is at a lower temperature than the prescribed temperature. In the present invention, for example, the temperature of the high-temperature high-pressure fluid used as a raw material can generally be set at a temperature that is 5 to 400° C. higher than the prescribed temperature, and the desired high-temperature high-pressure fluid can be produced by setting the temperature of the raw-material high-temperature high-pressure fluid at a temperature that is higher than the prescribed temperature (preferably in the range of 5 to 300° C., more preferably in the range of 5 to 250° C., and most preferably in the range of 5 to 200° C. higher than the prescribed temperature).

In the present invention, the setting of the mixture ratios of the high-pressure fluid which is at a higher temperature than the prescribed temperature and the high-pressure fluids which are at a lower temperature than the prescribed temperature is especially important for determining the reaction temperature; ordinarily, these mixture ratios are controlled by controlling the feeding rates or the linear velocities of the high-pressure fluid which is at a higher temperature than the prescribed temperature and the high-pressure fluids which are at a lower temperature than the prescribed temperature. In the present invention, the feeding rate of the high-pressure fluid (carrier fluid) which is at a higher temperature than the prescribed temperature and the feeding rate of the high-pressure fluids (high-pressure fluids of substrate) which are at a lower temperature than the prescribed temperature may ordinarily be values in the range of 10⁻³ to 10⁶ ml/min. The feeding rates used are preferably in the range of 10⁻³ to 10⁴ ml/min, more preferably in the range of 10⁻³ to 10³ ml/min, even more preferably in the range of 10⁻² to 10³ ml/min, and most preferably in the range of 10⁻¹ to 10² ml/min. In the present invention, where the feeding rate of the high-pressure fluid (carrier fluid) which is at a higher temperature than the prescribed temperature is designated as 1, the values of the feeding rates of the high-pressure fluids (high-pressure fluids of substrate) which are at a lower temperature than the prescribed temperature can be appropriately selected from values in the range of 0.0001 to 1. The values selected are preferably in the range of 0.001 to 1, more preferably in the range of 0.005 to 1, and most preferably in the range of 0.01 to 1.

In cases where the flow velocity is used, the apparent velocity fluctuates according to the size, shape, internal volume, cross-sectional area, length and the like of the reaction vessel even at the same feeding rate; accordingly, the use of linear velocity instead of the abovementioned feeding rate is usually easier to comprehend in engineering terms. In the present invention, linear velocities in the range of 10⁻⁶ to 10³ m/sec can ordinarily be used as the flow velocities of the high-pressure fluid (carrier fluid) which is at a higher temperature than the prescribed temperature and the high-pressure fluids (high-pressure fluids of substrate) which are at a lower temperature than the prescribed temperature. The linear velocities used are preferably in the range of 10⁻⁵ to 10³ m/sec, more preferably in the range of 10⁻⁴ to 10³ m/sec, even more preferably in the range of 10⁻³ to 10² m/sec, and most preferably in the range of 10⁻² to 10² m/sec. Ordinarily, the mixture ratio of the carrier high-pressure fluid which is at a higher temperature than the prescribed temperature and the substrate high-pressure fluids which are at a lower temperature than the prescribed temperature can also be expressed by the ratio of the linear velocities. In a case where the linear velocity of the carrier high-pressure fluid which is at a higher temperature than the prescribed temperature is designated as 1, values in the range of 0.0001 to 1 can ordinarily be appropriately selected as the linear velocities of the substrate high-pressure fluids which are at a lower temperature than the prescribed temperature. The values selected are preferably in the range of 0.001 to 1, more preferably in the range of 0.05 to 1, and most preferably in the range of 0.01 to 1.

In the present invention, a high-temperature high-pressure fluid at a desired prescribed temperature can be produced by mixing the high-temperature high-pressure fluid (carrier fluid) which is at a higher temperature than the prescribed temperature and the high-pressure fluids (high-pressure fluids of substrate) which are at a lower temperature than the prescribed temperature. Ordinarily, the time required in order to reach the prescribed temperature can be reduced to 5 seconds or less. However, the time required in order to reach the prescribed temperature is preferably a short time of 3 seconds or less, more preferably a short time of 1 second or less, and most preferably a short time of 0.5 seconds or less.

Ordinarily, in the present invention, a high-temperature high-pressure fluid set in the temperature range of 250 to 600° C. can be appropriately produced. Here, a high-temperature high-pressure fluid which is preferably in the set temperature range of 250 to 500° C., more preferably in the set temperature range of 300 to 500° C., and most preferably in the set temperature range of 300 to 450° C., can be produced. Meanwhile, in the present invention, a high-temperature high-pressure fluid can be produced with the pressure appropriately selected from a pressure range of 10 to 100 MPa. Here, a high-temperature high-pressure fluid can be produced preferably in the pressure range of 10 to 80 MPa, more preferably in the pressure range of 10 to 60 MPa, and most preferably in the pressure range of 15 MPa to 50 MPa.

In the present invention, a specified high-temperature high-pressure fluid can be produced using preferably water, acetonitrile, ethyl alcohol, acetone, dimethyl sulfoxide, 1,4-dioxane, N,N-dimethylformamide, tetrahydrofuran or the like as a high-temperature high-pressure fluid. However, the high-temperature high-pressure fluids that can be used in the present invention are not limited to these solvents. The solvents cited below may be used in appropriate combinations of one or more solvents. For example, pentane, hexane, heptane, cyclohexane, decalin, benzene, toluene, xylene, perfluorobenzene, fluorobenzene, hexafluorobenzene and the like may be cited as examples of hydrocarbons with a small polarity. Furthermore, benzonitrile and the like may be cited as examples of nitriles that have cyano groups.

Moreover, methanol, propanol, isopropanol, butanol, pentanol, cyclopentanol, hexanol, cyclohexanol, heptanol, cycloheptanol, octanol, cyclooctanol, nonanol, decanol, dodecanol, tridecanol, tetradeconal, heptadeconal, cycloheptanol, methoxyethanol, chloroethanol, trifluoroethanol, hexafluoropropanol, phenol, benzyl alcohol, ethylene glycol, triethylene glycol and the like may be cited as examples of alcohols that have hydroxy groups. Furthermore, ethyl acetate, methyl acetate, formic acid, acetic acid, dimethyl carbonate, diethyl carbonate, propylene carbonate and the like may be cited as examples of carboxylic acids, esters that are derivatives of carboxylic acids, carbonic acid or carbonic acid esters. Moreover, 2-butanone, 3-pentanone, diethyl ketone, methyl ethyl ketone, methyl propyl ketone, butyl methyl ketone, cyclohexanone, acetonphenone and the like may be cited as examples of ketones or aldehydes that have carbonyl groups. Furthermore, diglyme, diethyl ether, anisole and the like may be cited as examples of ethers.

Moreover, formamide, N-methylformamide, N,N′-dimethylacetamide, pyrrolidone, N-methylpyrrolidone, N,N′-dimethylethyleneurea, N,N′-dimethylpropyleneurea and the like may be cited as examples of amides or ureas that have amido groups. Furthermore, amines that have amino groups may also be used; examples of such amines include quinoline, triethylamine, tributylamine and the like. Moreover, sulfolane and the like may be cited as examples of sulfides or sulfoxides. Furthermore, hexamethylenephosphoric acid, phosphoric acid and the like may be cited as examples of phosphoric acids or phosphoric acid esters. Moreover, imidazole derivative salts which are ionic fluids, and halogen-containing hydrocarbons such as methylene chloride or the like may be cited. Solvents which can be used as high-temperature high-pressure fluids may comprise one or more solvents selected from the abovementioned solvents. Furthermore, high-temperature high-pressure fluids can be suitably produced by appropriately mixing these solvents, and these high-temperature high-pressure fluids can also be used in the production or measurement of compounds.

For example, the high-temperature high-pressure fluids obtained by means of the present invention reach the abovementioned prescribed temperature in a short time of 1 second or less, and can be used in a flow type reaction apparatus; for example, since a reaction can be performed in 2 seconds or less, such fluids can be effectively used in the synthesis or production of chemical compounds, the production of amino acids from protein raw materials, the synthesis of peptides from amino acids and the like. For example, in cases where a supercritical fluid is used in the present invention, synthesis reactions utilizing an acid catalyst function such as the production of lactams from oximes, e.g., the synthesis of ε-caprolactam from cyclohexanoneoxime by a Beckman transfer reaction, the production of pinacolin from pinacol by a pinacolin transfer reaction and the like, synthesis reactions utilizing an alkali catalyst function such as the production of alcohols and carboxylic acids from aldehydes by Cannizzaro's reaction and the like, oxidation reactions such as the production of phenol from benzene and the like, as well as the production of amino acid compounds from organic acids, the production of amino acids and lactams from lactone compounds, the production of lactams from amino acids and the like can be efficiently performed. Furthermore, the present invention can also be used in the production useful components by the decomposition or extraction of natural raw materials, such as the production of amino acids from natural protein raw materials such as corn, soybeans, fish meal and the like.

If the method of the present invention for the production of a high-temperature high-pressure fluid is used in flow type on-site measuring devices that utilize infrared spectroscopy, Raman spectroscopy, visible/ultraviolet light spectroscopy, nuclear magnetic resonance spectroscopy or the like, the interior of the measurement system can be placed in a high-temperature high-pressure state in a short time of (for example) 0.5 seconds or less. Thus, the method of the present invention can be used in the explication of the solvent characteristics of high-temperature high-pressure fluids, the interaction between substrates and solvents, the reaction methodes of substrates, reaction dynamics and the like.

Next, the high-temperature high-pressure reaction system of the present invention will be described with reference to FIG. 1, which shows one embodiment of this system. In terms o major constituent parts, the high-temperature high-pressure reaction system shown in FIG. 1 comprises six constituent parts. Specifically, this system is constructed from a carrier fluid feeding part which has a heater and a feeding pump, and which feeds the high-temperature high-pressure carrier fluid, a substrate injection part which feeds the low-temperature substrate high-pressure fluids, and which can inject these fluids into the high-temperature high-pressure carrier fluid, a high-temperature high-pressure reaction part which has a reaction vessel in which chemical reactions or the like are performed, a cooling part, a pressure control part which has a pressure regulating valve, and a sample recovery part which has a sample recovery vessel that recovers the reaction product. In the present invention, these constituent parts can be arbitrarily designed using appropriate means that have the abovementioned functions. Furthermore, in the reaction system of the present invention, optional means may be added in addition to the abovementioned means for feeding a high-pressure fluid which is at a higher temperature than the prescribed temperature into the reaction vessel as a carrier fluid and means for injecting one or more low-temperature substrate high-pressure fluids containing reactants into the reaction vessel.

In regard to the feeding pump used in the carrier fluid part, a single pump is ordinarily sufficient. However, the feeding rate can be accelerated by increasing the number of feeding pumps as necessary; alternatively, the temperature can be controlled by adding a fluid at a different temperature, or different fluids can be mixed. In regard to the heater that controls the heating of the carrier fluid, a single heater is sufficient; however, the temperature of the carrier fluid can be controlled more accurately by installing two or more heaters.

In cases where the reactant is a single reactant, a single pump is sufficient as the feeding pump used in the substrate injection part; however, in cases where there are two or more reactants, two or more feeding pumps may be used as necessary. Ordinarily, in most cases, a single reactant is dissolved in one substrate solution and used; however, two or more reactants may be dissolved in one substrate solution and used as necessary. For example, acids, alkalies, metal ions or the like may be added and used for the purpose of accelerating the reaction. Ordinarily, in most cases, these substances are dissolved and used in the substrate solution(s); alternatively, substances that do not react during the elevation of the temperature may also be used by being dissolved in the carrier water.

In the high-temperature high-pressure reaction system of the present invention, a pipe-form reaction vessel is ordinarily used in most cases; the reaction time can be varies by controlling the liquid feeding rate and the internal volume, cross-sectional area, length and the like of the reaction vessel. The reaction vessel may also be used with heat insulation provided by an adiabatic material so that the reaction temperature does not vary. There is no particular need to heat the reaction vessel; however, there is no objection to using the reaction vessel in a heated state.

After leaving the high-temperature high-pressure reaction part, the reaction solution is cooled in the cooling part so that the reaction temperature is lowered; then, the pressure is released in the pressure control part, and the sample can be collected and recovered in the sample recovery vessel in the recovery part. The reaction solution thus obtained is subjected to a solvent extraction process, column process or the like so that the desired substance is separated and purified, and this substance is used in products or the like.

In the present invention, it is most desirable that a supercritical fluid be used as the abovementioned high-temperature high-pressure fluid; however, sub-critical fluids may also be used. For example, in cases where water is used, this water may be used at a temperature of 374° C. or higher, and under a pressure of 22.1 MPa or greater. Furthermore, this can be suitably used in the production of chemical compounds at a temperature of 250° C. or higher and a pressure of 10 MPa or greater, which is the sub-critical fluid region.

In the present invention, the temperature can be caused to reach the prescribed reaction temperature in a short time of 5 seconds or less by setting the temperature of the carrier fluid beforehand at a temperature that is higher than the prescribed reaction temperature, and mixing this with a substrate solution at a temperature of for example 100° C. or lower. The prescribed temperature of the carrier fluid may conceivably fluctuate according to the size, volume and shape of the reaction vessel, the types, temperatures and pressures of the carrier fluid and substrate high-pressure fluids, the values of the feeding rate ratios of the respective fluids and the like. Generally, however, the temperature of the carrier fluid can be set at a temperature that is 5 to 400° C. higher than the prescribed reaction temperature, and it is advisable to perform the reaction with the prescribed temperature of the carrier fluid set at a temperature that is higher than the prescribed reaction temperature, preferably by a temperature in the range of 5 to 300° C., more preferably by a temperature in the range of 5 to 250° C., and most preferably in the range of 5 to 200° C.

In cases where ordinary reaction methods and reaction systems that have been used in the past are used, it is commonly recognized that side reactions such as hydrolysis reactions and the like occur under water heating conditions of 100 to 300° C. when a reaction is performed at a temperature of 300° C. or higher, especially in cases where the reactants are organic compounds, so that it is difficult to obtain the desired reaction product. In the high-temperature high-pressure reaction system of the present invention, since a specified high-temperature high-pressure state is reached in a short time of 5 seconds or less, there is almost no occurrence of side reactions such as hydrolysis reactions or the like that ordinarily occur under water heating conditions of 100 to 300° C. Accordingly, in the present invention, the substrate high-pressure fluids may be used at a temperature of 100° C. or lower. Since such substrate solutions are ordinarily prepared at room temperature in most cases, there is no objection to using such solutions “as is”.

In cases where the reactants are solid, these reactants are ordinarily dissolved in a solvent and used in the form of a substrate solution. The solvent used need not be the same as that of the carrier fluid; solvents that readily dissolve the reactants may be appropriately selected and used. Furthermore, in cases where the reactants are solutions, these reactants may be used “as is”, or may be used in a form in which these reactants are mixed with other solvents. Even in the case of reactants which are solid at room temperature, but which assume a liquid form at a temperature of 100° C. or less, e.g., in the case of substrates such as cyclohexanoneoxime, these substrates may be heated to a temperature of 100° C. or less and used as a molten liquid. Solid reactants may also be pulverized and used in the form of a slurry. Of course, in cases where reactants which are such that no side reactions occur under water heating conditions of 100 to 300° C. are used, there is no objection to using substrate solutions at a temperature of 100° C. or higher.

In the high-temperature high-pressure reaction method and corresponding reaction system of the present invention, the temperature of the reactants can ordinarily be elevated to the prescribed reaction temperature in a short time of 5 seconds or less by mixing the carrier fluid and the substrate high-pressure fluid(s) containing reactants. This temperature is elevated preferably in a short time of 3 seconds or less, more preferably in a short time of 1 second or less, and most preferably in a short time of 0.5 seconds or less.

In the high-temperature high-pressure reaction method and corresponding reaction system of the present invention, the reaction time is ordinarily a short time of 60 seconds or less; however, the reaction is performed preferably in a short time of 30 seconds or less, more preferably in a short time of 10 seconds or less, even more preferably in a short time of 5 seconds or less, and most preferably in a short time of 3 seconds or less.

In the high-temperature high-pressure reaction method and corresponding reaction system of the present invention, the reactants can ordinarily be appropriately reacted in a temperature of 250 to 600° C. However, the reactants are preferably reacted in a temperature range of 250 to 500° C., more preferably reacted in a temperature range of 300 to 500° C., and most preferably reacted in a temperature range of 300 to 450° C. Meanwhile, in the present invention, a reaction can be performed at a pressure appropriately selected from a pressure range of 10 to 100 MPa; however, the reaction is preferably performed in a pressure range of 10 to 80 MPa, more preferably performed in a pressure range of 15 to 60 MPa, and most preferably performed in a pressure range of 15 MPa to 50 MPa.

For example, specified high-temperature high-pressure fluids can be produced by appropriately used water, acetonitrile, ethyl alcohol, acetone, dimethyl sulfoxide, 1,4-dioxane, N,N-dimethylformamide, tetrahydrofuran or the like as the high-temperature high-pressure carrier fluid or low-temperature substrate high-pressure fluids used in the high-temperature high-pressure reaction method and corresponding reaction system of the present invention, and these fluids can be used in organic synthesis reactions and the like. However, the high-temperature high-pressure fluids that can be used in the present invention are not limited to these solvents. The solvents cited below may be used in appropriate combinations of one or more solvents. For example, pentane, hexane, heptane, cyclohexane, decalin, benzene, toluene, xylene, perfluorobenzene, fluorobenzene, hexafluorobenzene and the like may be cited as examples of hydrocarbons with a small polarity. Furthermore, benzonitrile and the like may be cited as examples of nitriles that have cyano groups.

Moreover, methanol, propanol, isopropanol, butanol, pentanol, cyclopentanol, hexanol, cyclohexanol, heptanol, cycloheptanol, octanol, cyclooctanol, nonanol, decanol, dodecanol, tridecanol, tetradeconal, heptadeconal, cycloheptanol, methoxyethanol, chloroethanol, trifluoroethanol, hexafluoropropanol, phenol, benzyl alcohol, ethylene glycol, triethylene glycol and the like may be cited as examples of alcohols that have hydroxy groups. Furthermore, ethyl acetate, methyl acetate, formic acid, acetic acid, dimethyl carbonate, diethyl carbonate, propylene carbonate and the like may be cited as examples of carboxylic acids, esters that are derivatives of carboxylic acids, carbonic acid or carbonic acid esters. Moreover, 2-butanone, 3-pentanone, diethyl ketone, methyl ethyl ketone, methyl propyl ketone, butyl methyl ketone, cyclohexanone, acetonphenone and the like may be cited as examples of ketones or aldehydes that have carbonyl groups. Furthermore, diglyme, diethyl ether, anisole and the like may be cited as examples of ethers.

Moreover, formamide, N-methylformamide, N,N′-dimethylacetamide, pyrrolidone, N-methylpyrrolidone, N,N′-dimethylethyleneurea, N,N′-dimethylpropyleneurea and the like may be cited as examples of amides or ureas that have amido groups. Furthermore, amines that have amino groups may also be used; examples of such amines include quinoline, triethylamine, tributylamine and the like. Moreover, sulfolane and the like may be cited as examples of sulfides or sulfoxides. Furthermore, hexamethylenephosphoric acid, phosphoric acid and the like may be cited as examples of phosphoric acids or phosphoric acid esters. Moreover, imidazole derivative salts which are ionic fluids, and halogen-containing hydrocarbons such as methylene chloride or the like may be cited. Solvents used as the high-temperature high-pressure carrier fluid or low-temperature substrate high-pressure fluids in the high-temperature high-pressure reaction method and corresponding reaction system of the present invention may comprise one or more solvents selected from the abovementioned solvents. Furthermore, the efficiency and selectivity can be improved by appropriately mixing such solvents, and these solvents can be appropriately used in the reaction and production of organic compounds.

In the present invention, desired high-temperature high-pressure fluids can be produced by mixing two or more high-pressure fluids at different temperatures in a flow system, and a high-temperature high-pressure fluid which is caused to reach a prescribed temperature in a short time of 5 seconds or less can be obtained by mixing a high-pressure fluid which is at a higher temperature than the prescribed temperature with high-pressure fluids that are at a lower temperature than the prescribed temperature. Furthermore, in the present invention, a highly efficient chemical compound producing system can be constructed using a flow type high-temperature high-pressure reaction method and corresponding reactions system that utilizes this high-temperature high-pressure fluid.

### BRIEF DESCRIPTION OF THE DRAWINGS

FIG. 1 shows one example of a flow type high-temperature high-pressure reaction system equipped with two water feeding pumps used in the present invention.

### DESCRIPTION OF THE PREFERRED EMBODIMENTS

Next, the present invention will be concretely described on the basis of examples; however, the present invention is not limited to these examples alone.

### EXAMPLE 1

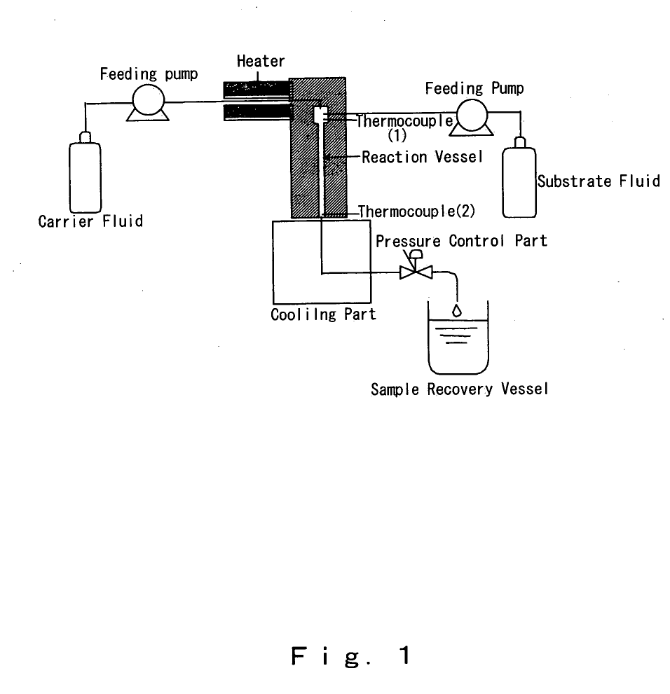

The production of high-temperature high-pressure water with a temperature of 420° C., a pressure of 40 MPa and a density of 0.4238 g/cm³ was tried using the flow type high-temperature high-pressure reaction apparatus shown in FIG. 1. The material of the reaction vessel was Hastelloy alloy C-276. The internal diameter of the reaction vessel was 0.325 mm and the length of the reaction vessel was 120 cm; accordingly, the volume of the reaction vessel was calculated as 0.0995 cm³. High-temperature high-pressure water with a temperature of 550° C. and a pressure of 40 MPa was prepared by using a heater to heat distilled water from which oxygen had been driven out by bubbling with nitrogen gas, and this was fed at a liquid feeding rate of 4.2 ml/min. The linear velocity was 8.43×10⁻¹ m/sec.

Meanwhile, high-pressure water at room temperature was prepared using room-temperature distilled water which had been similarly subjected to a deoxygenation treatment. This room-temperature high-pressure water at room temperature and at a pressure of 40 MPa was injected into the high-temperature high-pressure water at the inlet of the reaction vessel at a liquid feeding rate of 0.8 ml/min, and these two types of water were mixed. The linear velocity was 1.61×10⁻¹ m/sec. Accordingly, the liquid feeding rate of the high-temperature high-pressure water that was produced was 5.0 ml/min, and the linear velocity was calculated as 1.00×10⁰ m/sec. The temperature of the mixed solution measured by the thermocouple (1) installed at a distance of 1 cm from the inlet of the reaction vessel was 420° C. This value agreed with the temperature of 420° C. measured by the thermocouple (2) at the outlet of the reaction vessel; thus, it appeared that the temperature inside the reaction vessel was constant, and that the raw-material high-temperature high-pressure water and room-temperature high-pressure water were homogeneously mixed while passing through a distance of 1 cm in the reaction tube. Specifically, it appears that the temperature of the room-temperature high-pressure water was elevated from 25° C. to 420° C. as a result of short-time mixing, and that the temperature of the raw-material high-temperature high-pressure water conversely dropped from 550° C. to 420° C. In other words, it appears that mixing was completely accomplished within a short time of 0.005 seconds or less.

### EXAMPLE 2

The production of high-temperature high-pressure water with a temperature of 260° C., a pressure of 15 MPa and a density of 0.7964 g/cm³ was tried by performing an operation similar to that of Example 1. However, the conditions of use of the raw-material high-temperature high-pressure water and room-temperature high-pressure water used were altered as shown below.

Producing Conditions

Temperature and pressure of raw-material high-temperature high-pressure water: 355° C. and 15 MPa

Liquid feeding rate of raw-material high-temperature high-pressure water: 2.6 ml/min

Linear velocity of raw-material high-temperature high-pressure water: 5.22×10⁻¹ m/sec

Temperature and pressure of raw-material room-temperature high-pressure water: 25° C. and 15 MPa

Liquid feeding rate of raw-material room-temperature high-pressure water: 2.5 ml/min

Linear velocity of raw-material room-temperature high-pressure water: 50.2×10⁻¹ m/sec

The temperature of the mixed solution measured by the thermocouple (1) installed at the a distance of 1 cm from the inlet of the reaction vessel was 260° C. This agreed with the temperature of 260° C. measured by the thermocouple (2) at the outlet of the reaction vessel; thus, it appeared that the temperature inside the reaction vessel was constant, and that the raw-material high-temperature high-pressure water and room-temperature high-pressure water were homogeneously mixed while passing through a distance of 1 cm in the reaction tube. Specifically, it appears that the temperature of the room-temperature high-pressure water was elevated from 25° C. to 260° C. as a result of short-time mixing, and that the temperature of the raw-material high-temperature high-pressure water conversely dropped from 355° C. to 260° C. In other words, it appears that mixing was completely accomplished within a short time of 0.008 seconds or less. The liquid feeding rate of the produced high-temperature high-pressure water was 5.1 ml/min, and the linear velocity was calculated as 1.02×10⁰ m/sec.

### EXAMPLE 3

Using the continuous type high-temperature high-pressure reaction apparatus shown in FIG. 1, the continuous production of ε-caprolactam by a transfer reaction was tried using a cyclohexanoneoxime reagent (purity 97%) that was a product of Aldrich Chemical Co., Inc. under the following high-temperature high-pressure water conditions: temperature 400° C., pressure 40 MPa, density 0.5237 g/cm³. The material of the reaction vessel was alloy C-276. The internal diameter of the reaction vessel was 0.325 and the length of the reaction vessel was 120 cm; accordingly, the volume of the reaction vessel was calculated as 0.0995 cm³. The respective prepared liquids that were introduced were injected by means of high-pressure pumps. Carrier water with a temperature of 550° C. and a pressure of 40 MPa was prepared by heating distilled water from which solute oxygen had been driven out by bubbling with nitrogen gas, and this water was passed through at a liquid feeding rate of 3.7 ml/min. The linear velocity was 7.43×10⁻¹ m/sec.

Similarly, a substrate solution containing 21.9 mM cyclohexanoneoxime was prepared using distilled water that had been subjected to a deoxygenation treatment. The substrate solution with a temperature of room temperature and a pressure of 40 MPa was introduced into the carrier water at the inlet of the reaction vessel at a liquid feeding rate of 1.3 ml/min, and these solutions were mixed. The linear velocity was 2.61×10⁻¹ m/sec. The reaction temperature of the mixed solution measured by the thermocouple (1) installed at a distance of 1 cm from the inlet of the reaction vessel was 400° C. This agreed with the temperature of 400° C. measured by the thermocouple (2) at the outlet of the reaction vessel; thus, it appeared that the temperature was constant inside the reaction vessel, and that the carrier water and substrate solution were homogeneously mixed. The liquid feeding rate of the homogeneously mixed fluid was 5.0 ml/min, and the linear velocity was 1.00×10⁰ m/sec. The substrate concentration following mixing was 5.69 mM. The reaction time was 0.625 seconds. Accordingly, it appears that mixing was completely accomplished within a short time of 0.006 seconds or less.

When the aqueous solution recovered following the reaction was investigated using a high-speed liquid chromatography mass analysis apparatus, it was confirmed that ε-caprolactam was produced as the main product, and that 6-aminocaproic acid was produced as a by-product. In addition, unreacted cyclohexanoneoxime was detected. The content concentration of ε-caprolactam was 3.78 mM. The reaction yield was 66.4%. The content of 6-aminocaproic acid was 0.078 mM, and the reaction yield was 1.4%.

In conventional methods, cyclohexanone is produced from cyclohexanoneoxime, and the yield of ε-caprolactam is low. In the present invention, on the other hand, almost no production of cyclohexanone is seen; thus, it appears that side reactions occurring during the elevation of the temperature can be suppressed by reducing the temperature elevation time to a short time of 3 seconds or less, so that the yield of ε-caprolactam can be greatly increased in a reaction with a duration of 0.625 seconds. Thus, it appears that the use of the high-temperature high-pressure reaction system of the present invention makes it possible to perform a previously unknown novel organic reaction that proceeds in a short time of approximately 1 second.

### COMPARATIVE EXAMPLE 1

An experiment involving the synthesis of ε-caprolactam from cyclohexanoneoxime by means of a 3-minute batch type reaction method with the temperature set at 375° C. was performed by connecting thermocouples to a pipe-form reaction vessel made of SUS316 with an internal diameter of 8.7 mm and a length of 170 mm (internal volume 10.1 cm³), and attempting rapid temperature elevation using a molten salt bath. 3.5 g of distilled water and 0.5 g of cyclohexanoneoxime were placed in the reaction vessel, and the vessel was sealed in a nitrogen gas current. The temperature of this reaction vessel was elevated to the prescribed temperature by placing the reaction vessel in a molten salt bath that had been set at a temperature of 375° C. beforehand. The pressure at the reaction temperature was determined from the vapor pressure curve of the water on the basis of the internal volume and the amount and temperature of the water used. Following a reaction in the reaction vessel for 3 minutes at 375° C., the reaction was stopped by placing the reaction vessel in a cold water bath. The reaction pressure was 25 MPa, and the temperature elevation time to 375° C. was 29 seconds.

Following cooling, the product inside the reaction vessel was recovered using water and chloroform, and after the organic solvent layer was separated, the organic solvent was distilled away. The product was investigated by mass spectrometry, nuclear magnetic resonance spectrometry and gas-chromatographic analysis. As a result of analysis, it was found that the yield of ε-caprolactam was 14.7%, and that the yield of cyclohexanone was 45.8%. In the case of this batch synthesis method, since cyclohexanone which is the raw material of the cyclohexanoneoxime itself is produced in large quantities as a result of a hydrolysis reaction of the cyclohexanoneoxime, it appears that this method is unsuitable as an industrial process.

### EXAMPLE 4

The production of a high-temperature high-pressure fluid with a temperature of 375° C. and a pressure of 30 MPa was tried by performing an operation similar to that performed in Example 1. However, high-temperature high-pressure water was used as a raw-material fluid, and acetonitrile was used as a room-temperature high-pressure fluids; the conditions of use of these fluids were altered as shown below.

Producing Conditions

Temperature and pressure of raw-material high-temperature high-pressure water: 450° C. and 30 MPa

Liquid feeding rate of raw-material high-temperature high-pressure water: 3.9 ml/min

Linear velocity of raw-material high-temperature high-pressure water: 7.83×10⁻¹ m/sec

Temperature and pressure of raw-material room-temperature high-pressure acetonitrile: 25° C. and 30 MPa

Liquid feeding rate of raw-material room-temperature high-pressure acetonitrile: 1.1 ml/min

Linear velocity of raw-material room-temperature high-pressure acetonitrile: 2.21×10⁻¹ m/sec

The reaction temperature of the mixed solution measured by the thermocouple (1) installed at a distance of 1 cm from the inlet of the reaction vessel was 375° C. This agreed with the temperature of 375° C. measured by the thermocouple (2) installed at the outlet of the reaction vessel; thus, it appeared that the temperature inside the reaction vessel was constant, and that the raw-material high-temperature high-pressure water and room-temperature high-pressure acetonitrile were homogeneously mixed while passing through a distance of 1 cm in the reaction tube. A high-temperature high-pressure fluid comprising a water-acetonitrile system with a temperature of 375° C. and a pressure of 30 MPa containing 22 vol. % acetonitrile was obtained. Specifically, it appears that the temperature of the room-temperature high-pressure acetonitrile was elevated from 25° C. to 375° C. as a result of short-time mixing, and that the temperature of the high-temperature high-pressure water conversely dropped from 450° C. to 375° C. The high-temperature high-pressure fluid comprising a water-acetonitrile system showed behavior similar to that of water at a high temperature and high pressure, and if it is assumed that this fluid shows the average-weighted density of water and acetonitrile, the density is calculated as 0.5313 g/cm³ at a temperature of 375° C. and a pressure of 30 MPa. Thus, it appears that mixing was completely accomplished within a short time of 0.006 seconds or less. The liquid feeding rate of the high-temperature high-pressure fluid comprising a water-acetonitrile system was 5.0 ml/min, and the linear velocity was calculated as 1.00×10⁻¹ m/sec.

### EXAMPLE 5

The continuous production of ε-caprolactam by a transfer reaction of cyclohexanoneoxime as tried by performing a reaction using an operation similar to that performed in Example 3. However, a 354.0 mM substrate acetonitrile solution was prepared by dissolving the cyclohexanoneoxime in acetonitrile, and the reaction conditions were altered as shown below.

Reaction Conditions

Temperature and pressure of raw-material high-temperature high-pressure water: 500° C. and 40 MPa

Liquid feeding rate of raw-material high-temperature high-pressure water: 2.9 ml/min

Linear velocity of raw-material high-temperature high-pressure water: 5.82×10⁻¹ m/sec

Temperature and pressure of 354.0 mM substrate acetonitrile solution: 25° C. and 40 MPa

Liquid feeding rate of 354.0 mM substrate acetonitrile solution: 2.1 ml/min

Linear velocity of 354.0 mM substrate acetonitrile solution: 4.22×10⁻¹ m/sec

Temperature of reaction high-temperature high-pressure fluid: 350° C.

Pressure of reaction high-temperature high-pressure fluid: 40 MPa

Liquid feeding rate of reaction high-temperature high-pressure fluid: 5.0 ml/min

Linear velocity of reaction high-temperature high-pressure fluid: 1.00×10⁰ m/sec

The substrate concentration of the cyclohexanoneoxime at the time of mixing was 148.7 mM. The high-temperature high-pressure fluid comprising a water-acetonitrile system showed behavior similar to that of water at a high temperature and high pressure, and if it is assumed that this fluid shows the average-weighted density of water and acetonitrile, the density is calculated as 0.6107 g/cm³ at a temperature of 350° C. and a pressure of 40 MPa. The reaction time was 0.729 seconds, and since the temperature inside the reaction vessel was constant, it was inferred that mixing was completely accomplished within a short time of 0.007 seconds or less.

When the aqueous solution following the reaction was investigated using a high-speed liquid chromatography mass analysis apparatus, it was confirmed that ε-caprolactam was produced as the main product, and that 6-aminocaproic acid was produced as a by-product. The content concentration of ε-caprolactam was 130.0 mM, and the reaction yield was 87.4%. Meanwhile, the content of 6-aminocaproic acid was 18.7 mM, and the reaction yield was 12.6%. The conversion rate of cyclohexanoneoxime was 100%, and 6-aminocaproic acid is easily rearranged into ε-caprolactam; accordingly, the selectivity for ε-caprolactam in this reaction may be viewed as being substantially 100%.

### EXAMPLE 6

The production of a high-temperature high-pressure fluid with a temperature of 400° C. and a pressure of 30 MPa was tried by performing an operation similar to that performed in Example 1. However, high-temperature high-pressure water was used as a raw-material fluid and dimethyl sulfoxide was used as a room-temperature high-pressure fluid; the conditions of use of these fluids were altered as shown below.

Producing Conditions

Temperature and pressure of raw-material high-temperature high-pressure water: 540° C. and 30 MPa

Liquid feeding rate of raw-material high-temperature high-pressure water: 43 ml/min

Linear velocity of raw-material high-temperature high-pressure water: 8.63×10⁰ m/sec

Temperature and pressure of raw-material room-temperature high-pressure dimethyl sulfoxide: 25° C. and 30 MPa

Liquid feeding rate of raw-material room-temperature high-pressure dimethyl sulfoxide: 7 ml/min

Linear velocity of raw-material room-temperature high-pressure dimethyl sulfoxide: 1.41×10⁰ m/sec

The reaction temperature of the mixed solution measured by the thermocouple (1) installed at a distance of 1 cm from the inlet of the reaction vessel was 400° C. This temperature agreed with the temperature of 400° C. measured by the thermocouple (2) at the outlet of the reaction vessel; thus it appeared that the temperature inside the reaction vessel was constant, and that the raw-material high-temperature high-pressure water and room-temperature high-pressure dimethyl sulfoxide were homogeneously mixed while passing through a distance of 1 cm in the reaction tube. A high-temperature high-pressure fluid comprising a water—dimethyl sulfoxide system with a temperature of 400° C. and a pressure of 30 MPa containing 14 vol. % dimethyl sulfoxide was obtained. Specifically, it appears that the temperature of the room-temperature high-pressure dimethyl sulfoxide was elevated from 25° C. to 400° C. as a result of short-time mixing, and that the temperature of the raw-material high-temperature high-pressure water conversely dropped from 540° C. to 400° C.

The high-temperature high-pressure fluid comprising a water—dimethyl sulfoxide system showed behavior similar to that of water at a high temperature and high pressure, and if it is assumed that this fluid shows the average-weighted density of water and dimethyl sulfoxide, the density is calculated as 0.3630 g/cm³ at a temperature of 400° C. and a pressure of 30 MPa. Thus, it appears that mixing was completely accomplished within a short time of 0.004 seconds or less. The liquid feeding rate of the high-temperature high-pressure fluid comprising a water—dimethyl sulfoxide system was 50 ml/min, and the linear velocity was calculated as 1.00×10¹ m/sec.

### EXAMPLE 7

The production of a high-temperature high-pressure fluid with a temperature of 350° C. and a pressure of 30 MPa was tried by performing an operation similar to that performed in Example 1. However, high-temperature high-pressure water was used as a raw-material fluid, and ethyl alcohol was used as a room-temperature high-pressure fluid; the conditions of use of these fluids were altered as shown below.

Producing Conditions

Temperature and pressure of raw-material high-temperature high-pressure water: 410° C. and 30 MPa

Liquid feeding rate of raw-material high-temperature high-pressure water: 80 ml/min

Linear velocity of raw-material high-temperature high-pressure water: 1.61×10¹ m/sec

Temperature and pressure of raw-material room-temperature high-pressure ethyl alcohol: 25° C. and 30 MPa

Liquid feeding rate of raw-material room-temperature high-pressure ethyl alcohol: 20 ml/min

Linear velocity of raw-material room-temperature high-pressure ethyl alcohol: 4.02×10⁰ m/sec

The reaction temperature of the mixed solution measured by the thermocouple (1) installed at a distance of 1 cm from the inlet of the reaction vessel was 350° C. This temperature agreed with the temperature of 350° C. measured by the thermocouple (2) at the outlet of the reaction vessel; thus, it appeared that the temperature inside the reaction vessel was constant, and that the raw-material high-temperature high-pressure water and room-temperature high-pressure ethyl alcohol were homogeneously mixed while passing through a distance of 1 cm in the reaction tube. A high-temperature high-pressure fluid with a temperature of 350° C. and a pressure of 30 MPa comprising a water—ethyl alcohol system containing 20 vol. % ethyl alcohol was obtained. Specifically, it appears that the temperature of the room-temperature high-pressure ethyl alcohol was elevated from 25° C. to 350° C. as a result of short-time mixing, and that the temperature of the raw-material high-temperature high-pressure water conversely dropped from 410° C. to 350° C.

The high-temperature high-pressure fluid comprising a water—ethyl alcohol system showed behavior similar to that of water at a high temperature and high pressure, and if it is assumed that this fluid shows the average-weighted density of water and ethyl alcohol, the density is calculated as 0.6169 g/cm³ at a temperature of 350° C. and a pressure of 30 MPa. Thus, it appears that mixing was completely accomplished within a short time of 0.004 seconds or less. The liquid feeding rate of the high-temperature high-pressure fluid comprising a water—ethyl alcohol system was 100 ml/min, and the linear velocity was calculated as 2.01×10¹ m/sec.

### EXAMPLE 8

The production of a high-temperature high-pressure fluid with a temperature of 375° C. and a pressure of 30 MPa was tried by performing an operation similar to that performed in Example 1. However, high-temperature high-pressure water was used as a raw-material fluid, and acetone was used as a room-temperature high-pressure fluid; the conditions of use of these fluids were altered as shown below.

Producing Conditions

Temperature and pressure of raw-material high-temperature high-pressure water: 451° C. and 30 MPa

Liquid feeding rate of raw-material high-temperature high-pressure water: 0.4 ml/min

Linear velocity of raw-material high-temperature high-pressure water: 8.03×10⁻² m/sec

Temperature and pressure of raw-material room-temperature high-pressure acetone: 25° C. and 30 MPa

Liquid feeding rate of raw-material room-temperature high-pressure acetone: 0.1 ml/min

Linear velocity of raw-material room-temperature high-pressure acetone: 2.01×10⁻² m/sec

The reaction temperature of the mixed solution measured by the thermocouple (1) installed at a distance of 1 cm from the inlet of the reaction vessel was 375° C. This temperature agreed with the temperature of 375° C. measured by the thermocouple (2) at the outlet of the reaction vessel; thus, it appeared that the temperature inside the reaction vessel was constant, and that the raw-material high-temperature high-pressure water and room-temperature high-pressure acetone were homogeneously mixed while passing through a distance of 1 cm in the reaction tube. A high-temperature high-pressure fluid comprising a water-acetone system with a temperature of 375° C. and a pressure of 30 MPa containing 20 vol. % acetone was obtained. Specifically, it appears that the temperature of the room-temperature high-pressure acetone was elevated from 25° C. to 375° C. as a result of short-time mixing, and that the temperature of the raw-material high-temperature high-pressure water conversely dropped from 451° C. to 375° C. The high-temperature high-pressure fluid comprising a water-acetone system showed behavior similar to that of water at a high temperature and high pressure, and if it is assumed that this fluid shows the average-weighted density of water and acetone, the density is calculated as 0.6170 g/cm³ at a temperature of 350° C. and a pressure of 30 MPa. Thus, it appears that mixing was completely accomplished within a short time of 0.07 seconds or less. The liquid feeding rate of the high-temperature high-pressure fluid comprising a water—ethyl alcohol system was 0.5 ml/min, and the linear velocity was calculated as 1.00×10⁻¹ m/sec.

### EXAMPLE 9

The production of a high-temperature high-pressure fluid with a temperature of 375° C. and a pressure of 30 MPa was tried by performing an operation similar to that performed in Example 1. However, this operation was performed using the reaction vessel described below in place of the reaction vessel used in Example 1. The material of the reaction vessel used was alloy C-276; the internal diameter of the reaction vessel was 4.68 mm and the length of the reaction vessel was 200 mm. Accordingly, the volume of the reaction vessel was calculated as 3.440 cm³. Furthermore, high-temperature high-pressure water was used as a raw-material fluid, and 1,4-dioxane was used as a room-temperature high-pressure fluid. The conditions of use of these fluids were altered as shown below.

Producing Conditions

Temperature and pressure of raw-material high-temperature high-pressure water: 456° C. and 30 MPa

Liquid feeding rate of raw-material high-temperature high-pressure water: 3.9 ml/min

Linear velocity of raw-material high-temperature high-pressure water: 3.78×10⁻³ m/sec

Temperature and pressure of raw-material room-temperature high-pressure 1,4-dioxane: 25° C. and 30 MPa

Liquid feeding rate of raw-material room-temperature high-pressure 1,4-dioxane: 1.1 ml/min

Linear velocity of raw-material room-temperature high-pressure 1,4-dioxane: 1.07×10⁻³ m/sec

The reaction temperature of the mixed solution measured by the thermocouple (1) installed at a distance of 1 cm from the inlet of the reaction vessel was 375° C. This temperature agreed with the temperature of 375° C. measured by the thermocouple (2) at the outlet of the reaction vessel; thus, it appeared that the temperature inside the reaction vessel was constant, and that the raw-material high-temperature high-pressure water and room-temperature high-pressure 1,4-dioxane were homogeneously mixed while passing through a distance of 1 cm in the reaction tube. A high-temperature high-pressure fluid comprising a water—1,4-dioxane system with a temperature of 375° C. and a pressure of 30 MPa containing 22 vol. % 1,4-dioxane was obtained. Specifically, it appears that the temperature of the room-temperature high-pressure 1,4-dioxane was elevated from 25° C. to 375° C. as a result of short-time mixing, and that the temperature of the raw-material high-temperature high-pressure water conversely dropped from 456° C. to 375° C.

The high-temperature high-pressure fluid comprising a water—1,4-dioxane system showed behavior similar to that of water at a high temperature and high pressure, and if it is assumed that this fluid shows the average-weighted density of water and 1,4-dioxane, the density is calculated as 0.5620 g/cm³ at a temperature of 375° C. and a pressure of 30 MPa. Thus, it appears that mixing was completely accomplished within a short time of 1.2 seconds or less. The liquid feeding rate of the high-temperature high-pressure fluid comprising a water—1,4-dioxane system was 5.0 ml/min, and the linear velocity was calculated as 4.84×10⁻³ m/sec.

### EXAMPLE 10

The production of a high-temperature high-pressure fluid with a temperature of 375° C. and a pressure of 30 MPa was tried by performing an operation similar to that performed in Example 9. However, high-temperature high-pressure water was used as a raw-material fluid, and N,N-dimethylformamide was used as a room-temperature high-pressure fluid; the conditions of used of these fluids were altered as shown below.

Producing Conditions

Temperature and pressure of raw-material high-temperature high-pressure water: 450° C. and 30 MPa

Liquid feeding rate of raw-material high-temperature high-pressure water: 1.6 ml/min

Linear velocity of raw-material high-temperature high-pressure water: 1.55×10⁻³ m/sec

Temperature and pressure of raw-material room-temperature high-pressure N,N-dimethylformamide: 25° C. and 30 MPa

Liquid feeding rate of raw-material room-temperature high-pressure N,N-dimethylformamide: 0.44 ml/min

Linear velocity of raw-material room-temperature high-pressure N,N-dimethylformamide: 4.26×10⁻⁴ m/sec

The reaction temperature of the mixed solution measured by the thermocouple (1) installed at a distance of 1 cm from the inlet of the reaction vessel was 375° C. This temperature agreed with the temperature of 375° C. measured by the thermocouple (2) at the outlet of the reaction vessel; thus, it appeared that the temperature inside the reaction vessel was constant, and that the raw-material high-temperature high-pressure water and room-temperature high-pressure N,N-dimethylformamide were homogeneously mixed while passing through a distance of 1 cm in the reaction tube. A high-temperature high-pressure fluid comprising a water—N,N-dimethylformamide system with a temperature of 375° C. and a pressure of 30 MPa containing 21.6 vol. % N,N-dimethylformamide was obtained. Specifically, it appears that the temperature of the room-temperature high-pressure N,N-dimethylformamide was elevated from 25° C. to 375° C. as a result of short-time mixing, and that the temperature of the raw-material high-temperature high-pressure water conversely dropped from 450° C. to 375° C.

The high-temperature high-pressure fluid comprising a water—N,N-dimethylformamide system showed behavior similar to that of water at a high temperature and high pressure, and if it is assumed that this fluid shows the average-weighted density of water and N,N-dimethylformamide, the density is calculated as 0.5513 g/cm³ at a temperature of 375° C. and a pressure of 30 MPa. Thus, it appears that mixing was completely accomplished within a short time of 2.8 seconds or less. The liquid feeding rate of the high-temperature high-pressure fluid comprising a water—N,N-dimethylformamide system was 2.04 ml/min, and the linear velocity was calculated as 1.98×10⁻³ m/sec.

### EXAMPLE 11

The production of a high-temperature high-pressure fluid with a temperature of 375° C. and a pressure of 30 MPa was tried by performing an operation similar to that performed in Example 9. However, high-temperature high-pressure water was used as a raw-material fluid, and tetrahydrofuran was used as a room-temperature high-pressure fluid; the conditions of use of these fluids were altered as shown below.

Temperature and pressure of raw-material high-temperature high-pressure water: 455° C. and 30 MPa

Liquid feeding rate of raw-material high-temperature high-pressure water: 20.0 ml/min

Linear velocity of raw-material high-temperature high-pressure water: 1.94×10⁻² m/sec

Temperature and pressure of raw-material room-temperature high-pressure tetrahydrofuran: 25° C. and 30 MPa

Liquid feeding rate of raw-material room-temperature high-pressure tetrahydrofuran: 5.5 ml/min

Linear velocity of raw-material room-temperature high-pressure tetrahydrofuran: 5.33×10⁻³ m/sec

The reaction temperature of the mixed solution measured by the thermocouple (1) installed at a distance of 1 cm from the inlet of the reaction vessel was 375° C. This temperature agreed with the temperature of 375° C. measured by the thermocouple (2) at the outlet of the reaction vessel; thus, it appeared that the temperature inside the reaction vessel was constant, and that the raw-material high-temperature high-pressure fluid water and room-temperature high-pressure tetrahydrofuran were homogeneously mixed while passing through a distance of 1 cm in the reaction tube. A high-temperature high-pressure fluid comprising a water-tetrahydrofuran system with a temperature of 375° C. and a pressure of 30 MPa containing 21.6 vol. % tetrahydrofuran was obtained. Specifically, it appears that the temperature of the room-temperature high-pressure tetrahydrofuran was elevated from 25° C. to 375° C. as a result of short-time mixing, and the that temperature of the raw-material high-temperature high-pressure water conversely dropped from 455° C. to 375° C.

The high-temperature high-pressure fluid comprising a water-tetrahydrofuran system showed behavior similar to that of water at a high temperature and high pressure, and if it is assumed that this fluid shows the average-weighted density of water and tetrahydrofuran, the density is calculated as 0.5447 g/cm³ at a temperature of 375° C. and a pressure of 30 MPa. Thus, it appears that mixing was completely accomplished within a short time of 0.23 seconds or less. The liquid feeding rate of the high-temperature high-pressure fluid comprising a water-tetrahydrofuran system was 25.5 ml/min, and the linear velocity was calculated as 2.47×10⁻² m/sec.

### Industrial Applicability

In the present invention, as was described above in detail, a high-temperature high-pressure fluid can be produced by mixing two or more high-pressure fluids at different temperatures in a flow system, and a high-temperature high-pressure fluid whose temperature is caused to reach a prescribed temperature in 5 seconds or less can be obtained by mixing a high-pressure fluid which is at a temperature higher than the prescribed temperature with high-pressure fluid(s) which are at a temperature lower than the prescribed temperature. In the present invention, furthermore, the following special merits can be obtained by mixing and reacting a carrier fluid which is at a temperature higher than the prescribed reaction temperature with substrate solution(s) which are at a temperature of 100° C. or lower in a flow type high-temperature high-pressure reaction system: 1) the temperature elevation time to the prescribed reaction temperature can be reduced to a short time of 5 seconds or less, 2) side reactions such as hydrolysis reactions or the like can be suppressed, 3) a short-time reaction of 60 seconds or less can be efficiently performed, 4) desired chemical reactions can be selectively performed, and 5) a high-temperature high-pressure reaction method and corresponding reaction system in which novel organic synthesis reactions unknown in the past may proceed can be provided.

What is claimed is:

1. A method for the production of a high-temperature high-pressure fluid by mixing two or more high-pressure fluids at different temperatures in a flow system, comprises mixing a high-pressure fluid having a temperature higher than the prescribed temperature with a high-pressure fluid having a temperature lower than the prescribed temperature to cause the reactants to reach the prescribed reaction temperature in 5 seconds or less.

2. The method for the production of a high-temperature high-pressure fluid according to claim 1, wherein a high-temperature high-pressure fluid in a temperature range of 250 to 600° C. and a pressure range of 10 to 100 MPa is produced.

3. The method for the production of a high-temperature high-pressure fluid according to claim 1 or claim 2, comprises one or more high-temperature high-pressure fluids selected from a group comprising water, acetonitrile, ethyl alcohol, acetone, dimethyl sulfoxide, 1,4-dioxane, N,N-dimethylformamide and tetrahydrofuran are used and/or produced.

4. A high-temperature high-pressure reaction method for producing target substances by reacting one or more reactants in a high-temperature high-pressure fluid in a flow system, comprises feeding a high-pressure fluid having a higher temperature than the prescribed reaction temperature into a reaction vessel at a high speed as a carrier fluid, injecting one or more substrate high-pressure fluids which contain reactants and which have a lower temperature than the prescribed reaction temperature into the reaction vessel at a speed lower than the abovementioned speed, and mixing these fluids to cause the reactants to reach a prescribed reaction temperature in 5 seconds or less.

5. The high-temperature high-pressure reaction method according to claim 4, wherein the substances to be treated are reacted in a temperature range of 250 to 600° C. and a pressure range of 10 to 100 MPa.

6. The high-temperature high-pressure reaction method according to claim 4 or claim 5, wherein a high-pressure fluid at a temperature of 5 to 400° C. higher than the prescribed reaction temperature is used as the carrier fluid.

7. The high-temperature high-pressure reaction method according to any one of claims 4 through 6, wherein the temperature of the substrate high-pressure fluid containing the reactants is 100° C. or lower.

8. The high-temperature high-pressure reaction method according to any one of claims 4 through 7, wherein the linear velocity of the carrier fluid and/or substrate high-pressure fluid is 10⁻⁶ to 10³ m/sec.

9. The high-temperature high-pressure reaction method according to any one of claims 4 through 8, wherein the value of the linear velocity of the substrate high-pressure fluid containing the reactants is in the range of 0.0001 to 1, where the linear velocity of the carrier fluid is 1.

10. The high-temperature high-pressure reaction method according to any one of claims 4 through 9, wherein the feeding rate of the carrier fluid and/or substrate high-pressure fluid is 10⁻³ to 10⁶ ml/min.

11. The high-temperature high-pressure reaction method according to any one of claims 4 through 10, wherein the value of the feeding rate of the substrate high-pressure fluid containing reactants is a value in the range of 0.0001 to 1, where the feeding rate of the carrier fluid is 1.

12. The high-temperature high-pressure reaction method according to any one of claims 4 through 11, wherein the reaction time is 30 seconds or less.

13. The high-temperature high-pressure reaction method according to any one of claims 4 through 12, wherein one or more high-temperature high-pressure fluids selected from a group comprising water, acetonitrile, ethyl alcohol, acetone, dimethyl sulfoxide, 1,4-dioxane, N,N-dimethylformamide and tetrahydrofuran is used as a fluid.

14. A high-temperature high-pressure reaction system for use in the reaction method according to any one of claims 4 through 13, and for producing target substances by reacting one or more reactants in a high-temperature high-pressure fluid in a flow system, comprising:
means for feeding a high-pressure fluid having a higher temperature than the prescribed reaction temperature into the reaction vessel as a carrier fluid; and
means for injecting one or more low-temperature substrate high-pressure fluids containing reactants into the reaction vessel,
wherein the reactants are caused to reach the prescribed reaction temperature in 5 seconds or less by feeding a high-pressure fluid at a higher temperature than the prescribed reaction temperature into a reaction vessel at a high speed as a carrier fluid, injecting one or more substrate high-pressure fluids which contain reactants and which have a lower temperature than the prescribed reaction temperature into the reaction vessel at a speed lower than the abovementioned speed, and mixing these fluids.
